(19) United States
(12) Patent Application Publication
(10) Pub. No.: US 2016/0271318 A1
(43) Pub. Date: Sep. 22, 2016

(54) Method and apparatus for detecting hemolysis or for determining a correction factor to correct the influence of hemolysis on a measurement of hematocrit

(71) Applicant: FRESENIUS MEDICAL CARE DEUTSCHLAND GMBH, Bad Homburg (DE)

(72) Inventor: Christoph Wiktor, Gelnhausen (DE)

(21) Appl. No.: 15/033,508

(22) PCT Filed: Oct. 28, 2014
(86) PCT No.: PCT/EP2014/073160

§ 371 Date: Apr. 29, 2016

(30) Foreign Application Priority Data
Oct. 31, 2013 (DE) 10 2013 018 284.0

(51) Int. Cl. A61M 1/36; A61M 1/16; G01N 33/49

(52) U.S. Cl. CPC A61M 1/3681; G01N 33/49; A61M 1/16

(57) ABSTRACT

A method and an apparatus for detecting hemolysis or for determining a correction factor for correcting the influence of hemolysis on the hematocrit measurement, and an extracorporeal blood treatment apparatus with a device for detecting hemolysis. The invention is based on the implementation of two different optical measuring methods for determining hematocrit, wherein the hemolysis or the correction factor is established on the basis of the hematocrit values as detected by the two measuring methods. It has been found that the hematocrit values detected by the different measuring methods show that they are influenced with different intensity by the increasing concentrations of free hemoglobin in plasma consequent to hemolysis. A preferred embodiment envisions a reflection measurement as a first measuring method and a transmission measurement as a second measuring method. Detecting the hemolysis or establishing the correction factor is then achieved on the basis of forming the difference between the hematocrit values in the reflection and the transmission measurements. The apparatus includes a computing and analyzer unit 16 that is configured such that the hemolysis or the correction factor is established on the basis of the value as detected for hematocrit according to the first and the second measuring methods.

### CROSS REFERENCE TO RELATED APPLICATIONS

The present application is the national stage entry of International Patent Application No. PCT/EP2014/073160, filed on Oct. 28, 2014, the disclosure of which is expressly incorporated herein by reference in its entirety, and which claims priority to German Application DE 10 2013 018 284.0, filed on Oct. 31, 2013.

### FIELD OF INVENTION

The present invention relates to a method and apparatus for detecting hemolysis or for determining a correction factor for correcting the influence of hemolysis on a measurement of hematocrit. The present invention relates further to an apparatus for detecting hemolysis and for determining a correction factor for correcting the influence of hemolysis on the measurement of hematocrit, as well as an extracorporeal blood treatment apparatus with a device for detecting hemolysis.

### BACKGROUND

Hemolysis is among the possible complications of extracorporeal blood treatment. Hemolysis is understood as damage to the red blood cells (erythrocytes). Erythrocyte damage causes part of the hemoglobin to be present as free hemoglobin in blood plasma and part of the hemoglobin to be released to the haptoglobin that is present in blood. Both forms of hemoglobin in plasma are combined under the term plasma hemoglobin (PHb).

If the hemoglobin concentration in plasma exceeds that of free haptoglobin, clinical symptoms occur, including pain, inflammation and secretion of hemoglobin in the urine. Further symptoms are elevated blood pressure and pulse, an elevated thrombosis risk, difficulty swallowing and vomiting with traces of blood. If the hematocrit level drops steeply due to hemolysis, a poor oxygen supply to the organs as well as shortness of breath are the consequence.

When erythrocytes are damaged, aside from hemoglobin, the electrolyte potassium also escapes from the cells. Excess potassium concentrations in the extracellular space (plasma) disrupts normal signal conduction in nerves and muscles. Some of the consequences are severe cardiac arrhythmias, muscle paralysis, reduced reflexes and deeper than normal breathing. Since potassium is normally eliminated by the renal route, the effects of a hemolysis are tendentially more severe in patients who receive dialysis than in persons with normal kidney function. Correspondingly, detecting hemolysis is of particular significance when dialysis treatment is administered, which has the propensity of destroying erythrocytes in the extracorporeal blood circulation. Possible causes of erythrocyte damage are stenoses in the extracorporeal blood circulation due to production errors, kinked tubing or blocked cannulas and catheters.

A standard hemolysis detection method is an optical measurement involving centrifuged plasma samples in which the erythrocytes have formed a sediment, wherein the absorption of light in plasma is measured as different wavelengths. The proportion of free hemoglobin in plasma is determined on the basis of the measured value applying a formula that was established empirically.

WO 03/100416 A1 discloses an apparatus for determining extracellular hemoglobin concentrations that includes a transmitter and a receiver device that are disposed on both sides of a receptacle for a unit of stored blood. The transmitter and receiver unit measures the transmitted radiation that passes through the blood.

WO 2008/000433 A1 discloses a method and an apparatus for determining the concentration of blood components in the transparent hose line of an extracorporeal blood circulation that is filled with blood. A hose line of a tubing system is held during the measurement by being clamped between parallel, flat contact surfaces.

Replaceable units are known for blood treatment devices that comprise a plurality of components. These cartridges also have channels configured therein that carry the blood flow from the patient. A blood cartridge is known, for example, from WO 2010/121819.

Aside from measurements that operate on the basis of detecting the transmission of radiation in order to measure the hematocrit, other methods known in the art operate on the basis of detecting radiation that is scattered in the blood. Scattering is understood as the deflection of radiation due to interaction with a scattering center, wherein the scattering angle is defined as the angle by which the scattered particle is deflected. Scattering processes with a small scattering angle are referred to as forward scatter. Scattering processes with a scattering angle of between 90 degrees and 180 degrees are referred to as backward scatter (reflection). In a lateral scattering process, the scattering angle is 90 degrees.

### SUMMARY

An object of the present invention is to describe a non-invasive process for capturing hemolysis in whole blood that allows for a continuous hemolysis measurement.

A further object of the present invention is to provide a non-invasive method for determining a correction factor for correcting the influence of hemolysis on the measurement of hematocrit that will allow for arriving at a precise determination of hematocrit.

Further, it is another object of the present invention to provide an apparatus for the non-invasive, continuous detection of hemolysis or for determining a correction factor for a hematocrit measurement. Another object of the invention seeks to provide an extracorporeal blood treatment apparatus, particularly a dialysis instrument with a device for hemolysis detection.

The method and the apparatus according to the present invention are based on the implementation of two different optical measuring methods for arriving at the hematocrit value, wherein the hemolysis or the correction factor for correcting the influence of the hemolysis is determined on the basis of the two measuring methods for establishing the hematocrit value. The two measuring methods can be implemented simultaneously of within a short time of each other.

The whole blood is irradiated with radiation, particularly light, of a wavelength that is preferably in the visible range (380 nm to 780 nm). The first measuring method detects the radiation that emerges in relation to the incoming radiation from a first direction, while the second measuring method detects the radiation that emerges in relation to the incoming radiation from a second direction, wherein the first direction differs from the second direction. The intensity of the measured radiation is a function of the hematocrit; as the hematocrit increases, the intensity of the radiation decreases.

It has been found that the detected hematocrit values, which are established using the different measuring methods, are influenced with differing intensities by the increasing hemoglobin concentration in plasma. The determination of the hemolysis in the context of the method according to the present invention and the apparatus according to the present invention is based on the fact that an increasing hemoglobin concentration in the first measuring method will result in an increase or decrease, respectively, of the measured hematocrit value and to a decrease or increase, respectively, of the hematocrit value in the second measuring method.

Instead of determining the hemolysis rate, the method and apparatus according to the present invention also allow for determining a correction factor that can be applied for correcting the influence of the hemolysis on the measurement of hematocrit with the known hematocrit measuring methods not taking into account the influence of the hemolysis. Typically, it is presumed therein that, with the increasing concentration of free hemoglobin in plasma and/or increasing hemolysis rate, in known hematocrit measuring methods, the error of the measurement will increase. A correction factor is also understood as a correction value, meaning an absolute amount by which the measured value is increased or decreased.

The apparatus for determining the correction factor according to the present invention can be a structural assembly of the actual hematocrit measuring device.

In a preferred embodiment of the present invention, the hemolysis or the correction factor is determined based on the difference of the hematocrit value that was established according to the first and the second measuring methods. It has been found that the hemolysis increases with the increasing difference between the two measured hematocrit values. Consequently, the difference of the measured values is an order of magnitude for reflecting the hemolysis.

A further particularly preferred embodiment provides for determining the hemolysis rate on the basis of a preset relationship between the difference of the hematocrit values as determined according to the first and the second measuring methods and the hemolysis rate. Varying differences of hematocrit values can each be assigned a certain hemolysis rate. The empirically established value pairs can be stored in a memory. However, the hemolysis rate can also be calculated on the basis of a preset function that specifies the relationship between the difference values and the hemolysis rate.

The measuring methods can differ from each other in the way they detect the transmitted or scattered radiation. Forward scatter, backward scatter (reflection) or lateral scatter can be detected as scattered radiation.

Experiments have shown that, when using a transmission measurement, with the increasing concentration of free hemoglobin in plasma, smaller hematocrit values are measured than when a measurement of scatter is employed. When using a scatter-type measurement, different dependences result between the measured hematocrit value and the concentration of free hemoglobin in plasma. Reflection measurements show the most pronounced dependent relationships. While the measured hematocrit values decrease in transmission measurements when the concentration of free hemoglobin in plasma increases, the hematocrit values that are measured by a reflection measurement increase when the hemoglobin concentration in plasma increases. For all measuring methods, it has been possible to establish a linear relationship between the measured hematocrit values and plasma hemoglobin concentrations.

An especially preferred embodiment of the present invention provides for a reflection measurement as the first measuring method and a transmission measurement as the second measuring method. The hemolysis is detected based on the difference established from hematocrit values that were ascertained in the reflection and transmission measurements.

The apparatus according to the present invention includes a transmitting and a receiving unit for irradiating whole blood and for receiving the radiation that emerges from the whole blood or that is reflected by the same according to any one of the two measuring methods, particularly measuring transmitted and reflected radiation. Furthermore, the apparatus according to the present invention includes a computing and analyzer unit that is configured such that the hemolysis or the correction factor are determined on the basis of the hematocrit value that is detected based on the first and the second measuring method. The computing and analyzer unit is preferably a data processing unit (microprocessor) that executes a data processing software program.

In one preferred embodiment, the apparatus according to the present invention includes a unit with a receptacle for receiving by way of a clamping action a hose line of an extracorporeal blood circulation. An alternate embodiment provides for a unit for mounting a cartridge that includes a channel inside which the blood flows.

The transmitting and receiving unit includes one or a plurality of transmitters and one or a plurality of receivers that are disposed on one of the sides of the receiving unit. In a first alternate embodiment, the transmitting and receiving unit includes only one transmitter and two receivers, while, in the second alternate embodiment, it includes two transmitters and only one receiver. While, in the first embodiment, the radiation of the single transmitter can be received by both receivers simultaneously from different directions, in the second embodiment, the two transmitters must be alternately switched on and/or off to accommodate the only one receiver to be able to receive the radiation from different directions. However, it is also possible for the transmission and receiving unit to include respectively one transmitter and one receiver for each of the two measuring methods. The transmitter and receiver therein can each comprise a single or a plurality of LEDs and/or photo diodes.

The device according to the present invention can constitute a separate structural assembly or a component of an extracorporeal blood treatment apparatus, particularly a dialysis device. If the apparatus according to the present invention is a component of the blood treatment apparatus, it is possible for the computing and analyzer unit of the apparatus according to the present invention to also be a structural component of the central data processing unit of the blood treatment apparatus.

The apparatus according to the present invention is preferably used for measuring the hemolysis rate in the venous blood line that leads from the changing unit of the extracorporeal blood treatment apparatus to the patient, because, due to the mechanical damage suffered by the erythrocytes in the extracorporeal circulation, the level of damage is highest, when the blood enters the patient after running through the extracorporeal circulation. However, a measurement in the arterial blood line is possible as well. Combining both measurements allows for monitoring the totality of the extracorporeal blood circulation.

An embodiment of the present invention will be described in detail below based on the drawings.

### BRIEF DESCRIPTION OF THE DRAWINGS

FIG. 1 is a representation of an apparatus for the extracorporeal blood circulation with a device for detecting hemolysis, seen in a very simplified and schematic depiction.

FIG. 2A is a representation of a partial view of the measurement unit of the apparatus for detecting hemolysis with a transmitter and a receiver for detecting scattered radiation, seen in a simplified depiction.

FIG. 2B is a representation of a partial view of the measurement unit with a transmitter and a receiver for detecting transmitted radiation, seen in a simplified depiction.

FIG. 2C is a representation of a partial view of an alternate embodiment of the measurement unit with a transmitter and a receiver for detecting scattered radiation, seen in a simplified depiction.

FIG. 3 is a representation of a depiction of the principle as embodied in the transmitting and receiving units for measuring the radiation according to different measuring methods.

FIG. 4 is a representation of the intensity of the measured signal as a function of hematocrit.

FIG. 5 A to FIG. 5 D are representations of the measured hematocrit signal using different measuring methods.

FIG. 6 is a representation of the hematocrit value from the hemoglobin concentration in plasma established by different measuring methods.

FIG. 7 is a representation of the function of the differences of the hematocrit values and hemolysis rates as established by a reflection and transmission measurements.

FIG. 8 is a representation of a first embodiment of a measurement apparatus for reflection and transmission measurements.

FIG. 9 is a representation of a second embodiment of a measurement apparatus for reflection and transmission measurements.

### DETAILED DESCRIPTION

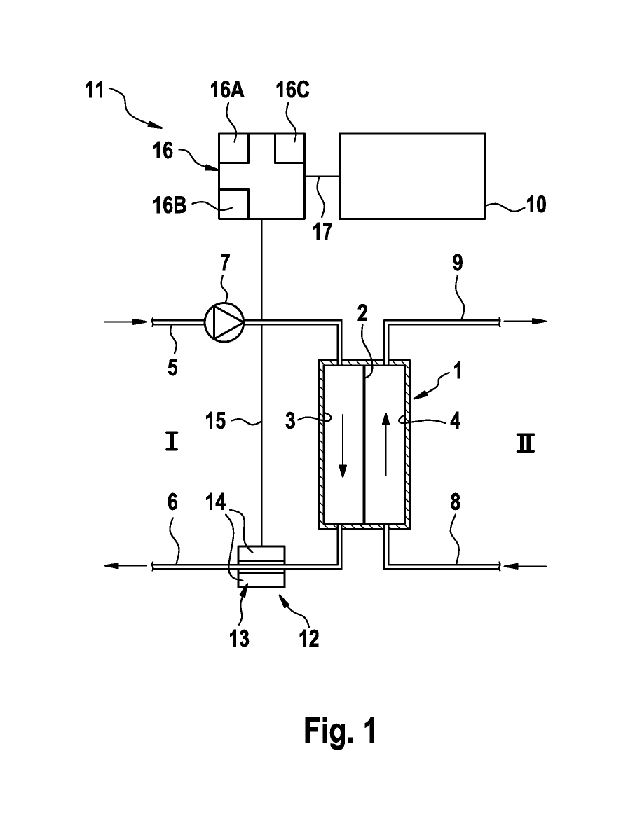

FIG. 1 only shows the components that are needed according to the invention for operating an apparatus for extracorporeal blood treatment, particularly a dialysis device, seen in a very simplified schematic depiction. The extracorporeal blood treatment apparatus includes an exchange unit, for example a dialyzer or filter 1 that is subdivided into a blood chamber 3 and a dialysis fluid chamber 4 by a semi-permeable membrane 2. An arterial blood supply line 5 leads from the patient to the blood chamber of the dialyzer 1, while a venous blood return line 6 branches off from the blood chamber leading to the patient. A blood pump 7 that is disposed in the arterial blood line 5 pumps the blood in the extracorporeal blood circulation 1.

The dialyzer fluid system II of the dialysis device is only hinted at in the drawing. It comprises a dialysis fluid supply line 8 leading to the dialysis fluid chamber 4 and a dialysis fluid discharge line 9 that branches off from the dialysis fluid chamber 4 of the dialyzer 1. The arterial and venous blood line 5, 6 are hose lines that are at least partially permeable for electromagnetic radiation, particularly light.

Moreover, the blood treatment apparatus includes a central control unit 10 that controls the individual components, for example the blood pump 7. In the presently shown embodiment, the apparatus 11 for determining the hemolysis is a structural component of the blood treatment apparatus, such that the same is able to utilize components that are present anyway as part of a blood treatment apparatus.

The apparatus 11 for detecting hemolysis or the correction factor includes a measuring unit 12 comprising a unit 13 into which a hose line of the extracorporeal blood circulation can be fitted, particularly the venous blood line 6. The measuring unit 12 comprises further a transmitter and receiver unit 14 for coupling radiation in and out.

A data line 15 connects the measuring unit 12 to the computing and analyzer unit 16. The computing and analyzer unit 16 is able to exchange data with the central control unit 10 of the blood treatment apparatus via a line 17.

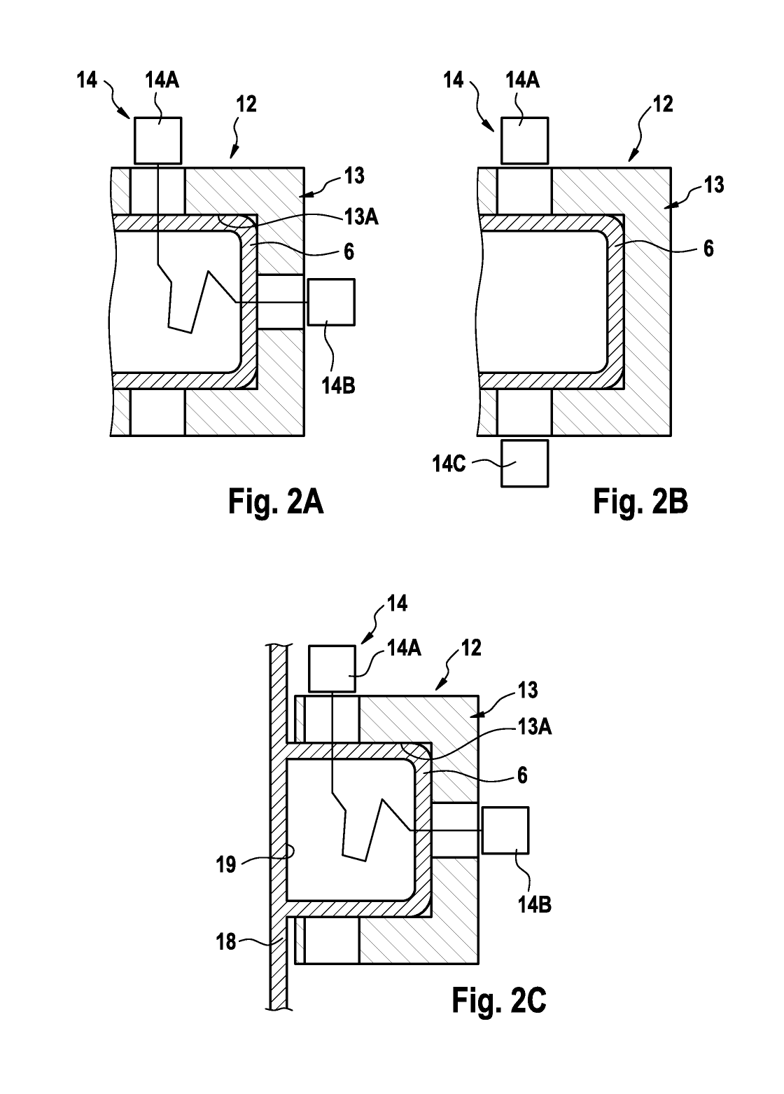

FIG. 2A shows a partial view of the measuring unit 12, seen as a simplified cut representation. The measuring unit 12 includes a unit 13 with a receptacle 13A into which the hose line 6 is clamped. The receptacle 13A includes four flat contact surfaces that are disposed at right angles relative to each other, and the hose line rests there against. FIG. 2A depicts only a single transmitter 14A and a single receiver 14B of the transmitter and receiver unit 14. The radiation that is emitted by the transmitter 14A passes through the hose line and into the blood that flows in the hose line 6, wherein the radiation emerging from the blood traverses through the hose line and contacts the receiver 14B. Due to the fact that the axes of the transmitter and receiver 14A, 14B are disposed with a right angle, the receiver receives the scattered radiation. For the detection of the transmitted radiation, the transmitter and the receiver 14A, 14C are disposed opposite each other on a shared axis, as depicted in FIG. 2B.

FIG. 2C shows an alternate embodiment of the measuring unit 12 that is intended for a blood cartridge 18, where the blood does not flow inside a hose line but a blood channel 19, which is configured inside the cartridge. The part of the blood cartridge 18 with the channel 19 is made of a transparent material. The measuring unit 12 includes, for example, a unit 13 that is open on one side and where the cartridge 18 can be fastened or that can be fastened to the cartridge. The measuring unit 12 and cartridge 18 thus constitute separate units, wherein the measuring unit 12 is a component of the blood treatment apparatus, and wherein the cartridge 18 can be exchanged.

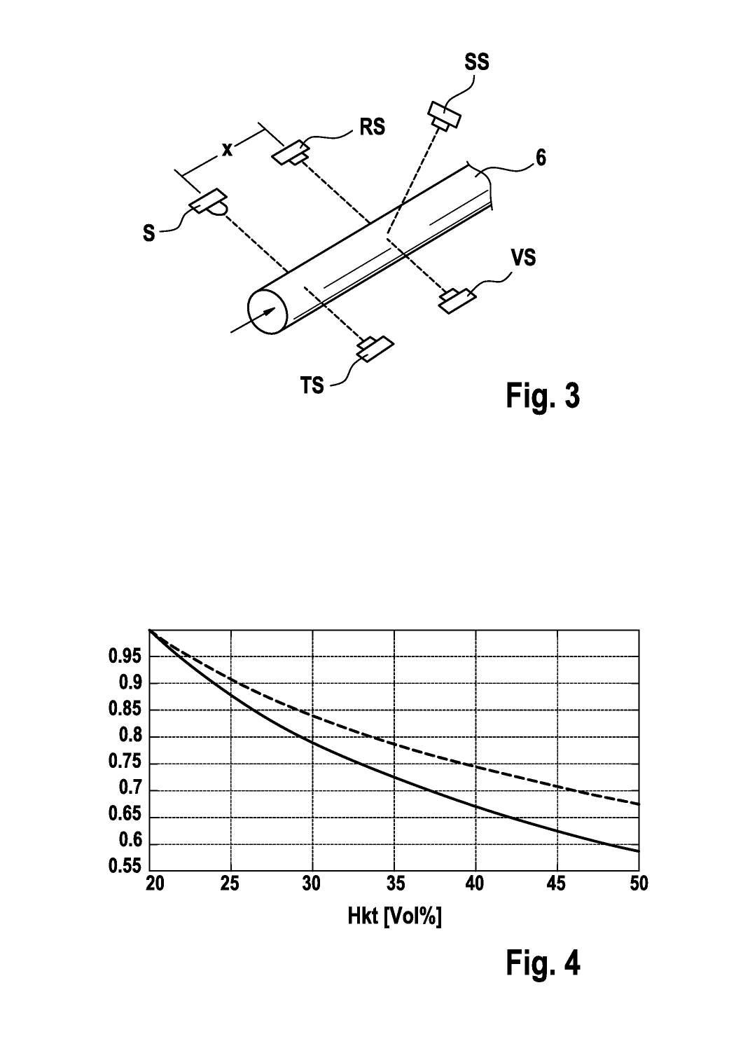

FIG. 3 shows a representation of the principle that is embodied in a measuring apparatus with a transmitter and receiver unit 14 comprising a plurality of transmitters and receivers to be able to determine the hematocrit value by way of different measuring methods. Due to the fact that the individual measuring methods are known from the prior art, we shall describe only the operative principle of the measurement apparatus.

The blood flows inside the transparent hose line 6 in the unit 13, which in clamped in the unit 13 of the device for determining hemolysis, not shown in FIG. 3. The measurement apparatus for the transmission measurement includes a transmitter S and a receiver that are disposed on both sides of the hose line on a shared axis facing each other. The receiver for the detection of the transmitted radiation is designated as TS. The axis of the transmitter S and receiver TS extends at a right angle relative to the longitudinal axis of the hose line 6. The light of the transmitter S, which propagates in the direction of the axis and meets the blood that flows inside the hose line, is received by the receiver TS. The receiver TS supplies a measured signal that is proportionate to the intensity of the light and that is analyzed in the computing and analyzer unit 16. Lambert-Beer's law describes the relationship between the intensity of the incoming and emerging light by which the hematocrit value is calculated in the computing and analyzer unit 16.

For the detection of the scattered radiation (scattered light measurement), the measurement apparatus includes three further receivers. The receiver for the detection of the backward scatter (reflection) is designated as RS, the receiver for detecting the forward scatter is designated as VS, and the receiver for detecting the lateral scatter is designated as SS. The receivers RS and VS are disposed at the distance x relative to the transmitter S and receiver TS for the transmission measurement. The transmitter S and the receiver TS for the transmission measurement and the receiver RS and VS for the detection of the backward scatter and forward scatter are arranged inside a plane through which the longitudinal axis of the hose line 6 extends. The receiver RS for detecting the reverse scatter, the receiver VS for detecting the forward scatter and the receiver SS for detecting the lateral scatter are arranged inside a plane that is perpendicular relative to the former plane. For the detection of the lateral scatter, the spacing x can also be zero.

The wavelength of the radiation that is emitted by the transmitter S, particularly the emerging light, is preferably not in the visible range of 380 nm to 780 nm. The transmitter is preferably a narrow-band LED with a peak wavelength that is at 805 nm. The receivers are photodiodes.

FIG. 4 shows a change of the intensity of the signal of the receiver SS (relative measurement signal (x=0)) for the lateral scatter as a function of the hematocrit Hkt in vol %, wherein an LED with a small dispersion angle of 30 degrees and a large dispersion angle of 110 degrees is used. The measurement signal of the LED (SMD LED) is depicted with a dispersion angle of 30 degrees as a solid line, and the measurement signal of the LED (wide angle LED (5 mm)) with a dispersion angle of 110 degrees as a perforated line. It is shown that, using an LED with a small dispersion angle, with increasing hematocrit, the change of the measurement signal is greater than when using an LED that has a large dispersion angle. Consequently, to increase sensitivity, the dispersion angle of the LED should be small.

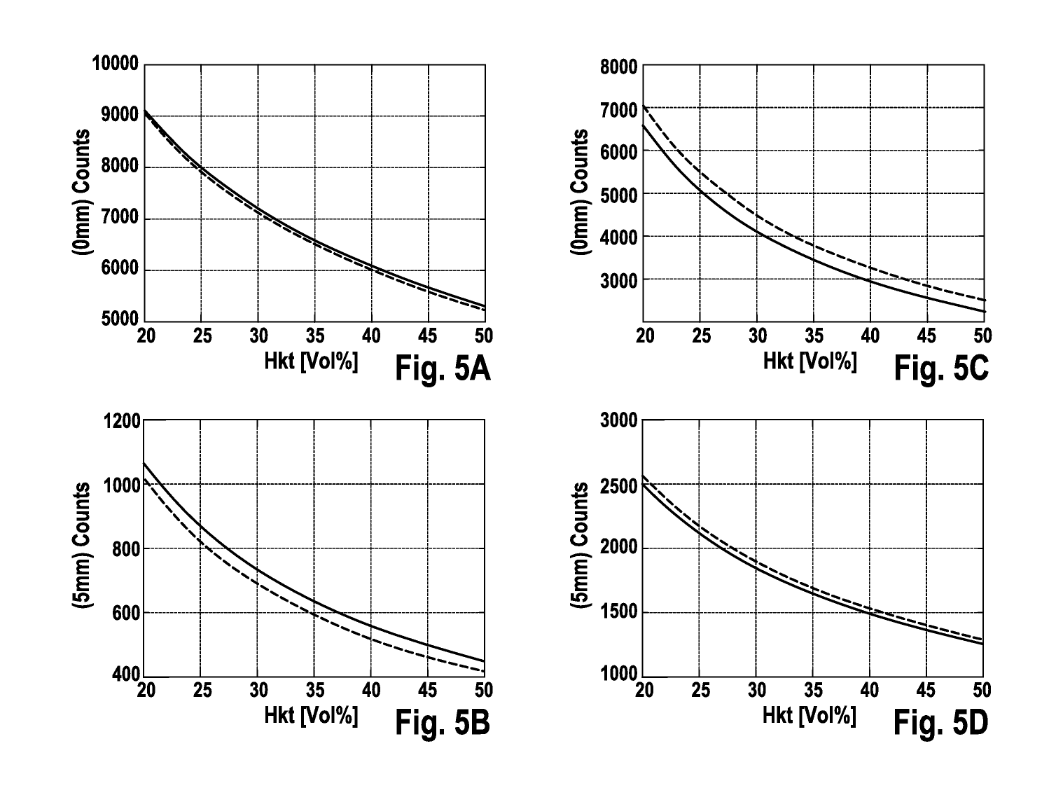

FIGS. 5A to 5D show the dependent relationship of the intensity of the radiation as measured with the different measuring methods [Counts] and hematocrit Hkt [vol %], wherein the measured values for human blood are depicted in a solid line and those for cattle blood in a perforated line. It is seen for all measuring methods that the intensity of the radiation decreases, when the hematocrit increases.

FIG. 5A shows the dependent relationship of the intensity of the radiation and the hematocrit for the measuring method that provides for detecting the lateral scatter (lateral scatter in counts), wherein the spacing is x=0. FIG. 5B shows the dependent relationship of the lateral scatter (lateral scatter in counts) and the hematocrit with a spacing of x=5 mm. FIG. 5C shows the dependent relationship of the intensity of the transmission measurement (x=0 mm) and FIG. 5D the dependent relationship of the intensity of the scattered radiation hematocrit Hkt as measured by the reflection measurement (x=5 mm). Varying dependent relationships emerge for the radiation intensity relative to hematocrit with regard to the different measuring methods (lateral scatter, transmitted, reflected).

The hematocrit value that is determined by means of different measuring methods is a function of the concentration of free hemoglobin in plasma due to hemolysis. Different dependent relationships emerge for the different measuring methods. However, the relationship between hematocrit and the concentration of free hemoglobin is always linear.

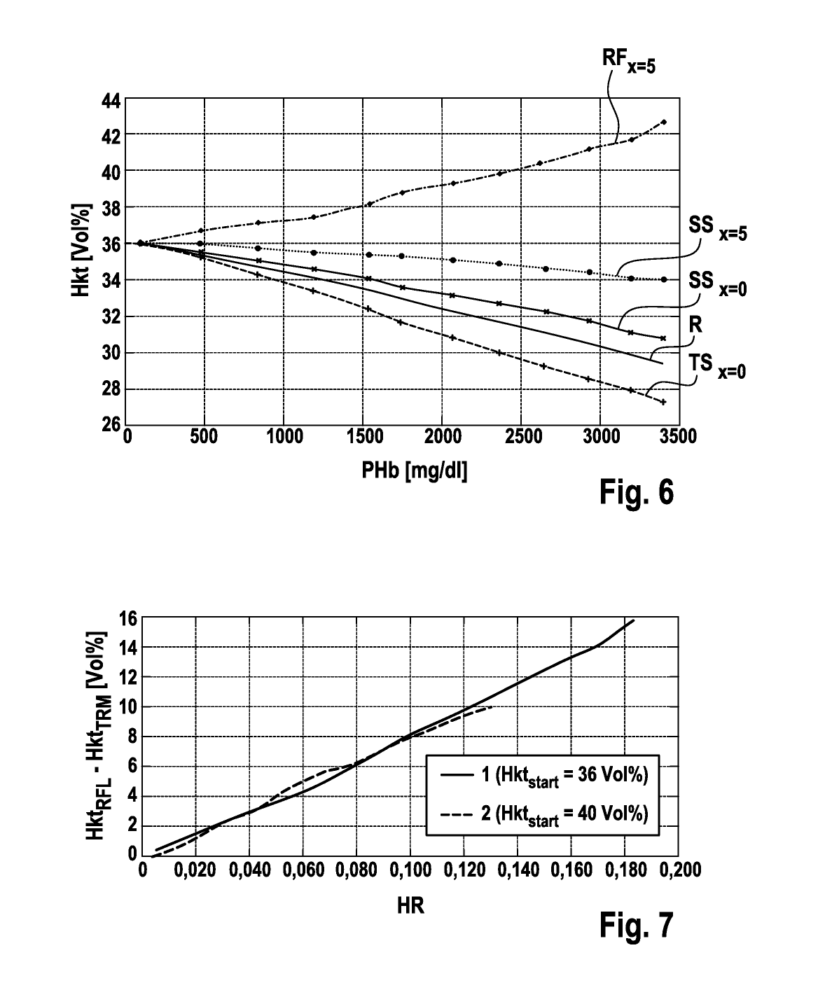

The dependent relationship between hematocrit and the concentration of free hemoglobin in plasma is shown for different measuring methods in FIG. 6. The graphics in FIG. 6 are referenced as follows:

RF: Reference value
SSₓ₌₀ Lateral scatter (x=0 mm),
TSₓ₌₀ Transmission (x=0 mm),
SSₓ₌₅ Lateral scatter (x=5 mm),
Rₓ₌₅ Reflection (x=5 mm).

Hematocrit HKt in vol % is plotted on the y-axis; the concentration K of free hemoglobin in plasma in mg/dl is plotted on the x-axis.

During the reflection measurement, the measured hematocrit values increase parallel to the increasing concentration of free hemoglobin in plasma while, during the measurement of the lateral scatter and transmission, the measured hematocrit values decrease parallel to the increasing plasma hemoglobin concentration. The most pronounced decrease of the measured hematocrit values can be seen in the transmission measurement. It is seen that the scattering measurement overestimates the hematocrit with increasing hemoglobin concentrations in plasma, while, in the transmission measurement, the hematocrit is underestimated. The reflection measurement reacts with the greatest sensitivity to the increasing hemolysis.

In the present embodiment, hematocrit is determined by two different measuring methods. The first measuring method includes a reflection measurement and the second measuring method a transmission measurement. These two measuring methods show the greatest dependence between the measured hematocrit value and the concentration of free hemoglobin in plasma.

The computing and analyzer unit 16 calculates the difference between the hematocrit value Hkt_{RFL} as measured by the reflection measurement and the hematocrit value Hkt_{TRM} as measured by the transmission measurement, wherein conclusions as to the presence of hemolysis and the amount of the hemolysis rate are drawn based on the difference of the two measured values. The conclusion that an increase of the hemolysis rate has occurred is based on the increase of this difference.

The hemolysis rate HR is an order of magnitude without dimension for the hemolysis determination that is independent of hematocrit. It is calculated from HR=(100%-Hkt)*PHb/GHb, wherein PHb is the hemoglobin concentration in plasma, and GHb is the total hemoglobin concentration in whole blood.

FIG. 7 demonstrates that there exists a for the most part linear relationship between the difference Hkt_{RFL}−Hkt_{TRM} of the measured values Hkt_{RFL}, Hkt_{TRM} for hematocrit and the hemolysis rate. The measured values were established in two experiments. The graphic that is shown in a solid line shows the dependence of the difference of the measured hematocrit values from the hemolysis rate for a starting value of hematocrit of 36 vol %; and the graphic drawn by the perforated line stands for a starting value of hematocrit of 40 vol %. Both graphics can be approximated by a straight line.

The individual measured values for the difference Hkt_{RFL}−Hkt_{TRM} and the associated hemolysis rates HR are stored in a memory of the computing and analyzer unit 16. The computing and analyzer unit 16 reads out of the memory 16A for the detected difference between the hematocrit values of the hemolysis rate HR. For example, a difference of measured values of 8 vol % results in a hemolysis rate HR of 100. However, it is also possible for an equation to reside in the computing and analyzer unit 16 that describes the dependent relationship of the difference of the hematocrit values and the hemolysis rate that is used for calculating the hemolysis rate as a function of the difference of the measured hematocrit values.

The hemolysis rate is represented on a display unit 16B of the device 11 for hemolysis detection. The device can include an alarm unit 16C that outputs an alarm when a preset hemolysis rate is exceeded. When a preset hemolysis rate is exceeded, it is also possible to generate a control signal that is received by the central control unit 10 of the blood treatment apparatus via line 17, such that it is possible to engage with the machine control of the blood treatment apparatus.

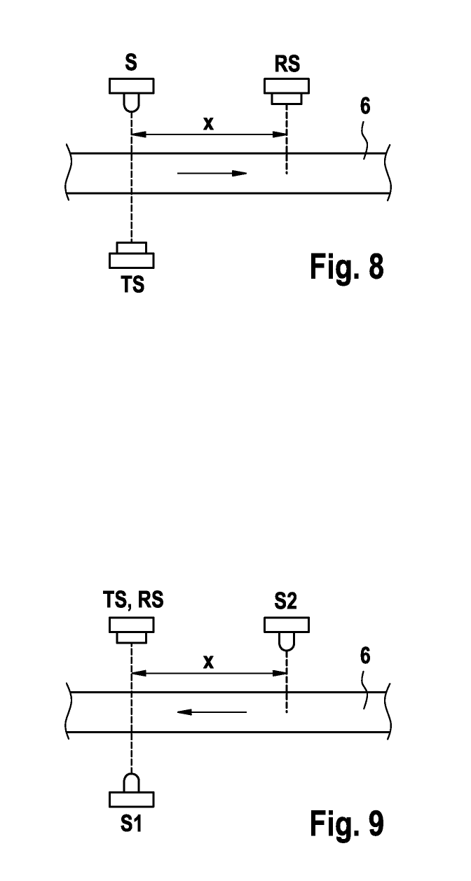

The preferred embodiment of the invention only utilizes reflection and transmission measurements. FIG. 8 shows a first alternate embodiment of the measurement apparatus for the reflection and transmission measurements. This embodiment corresponds to the embodiment as shown in FIG. 3, wherein the receivers VS and SS for the forward and lateral scatter have been omitted. Corresponding parts are therefore identified by the same reference signs. The measurement apparatus of FIG. 8 allows both receivers TS and RS to measure reflected and/or transmitted radiation simultaneously. FIG. 9 shows an alternate embodiment that includes a measurement path for the transmission measurement with a transmitter S1 and a receiver TS, RS that is also used for the reflection measurement. A second transmitter S2 is provided for the reflection measurement that is disposed, observing a spacing x of the measurement path, for the transmission measurement. This measurement apparatus does not allow for simultaneous but only for alternate measurements using the two measuring methods. For the transmission measurement, the computing and analyzer unit 16 activates the transmitter S1 for the transmission measurement and deactivates the transmitter S2 for the reflection measurement, while the transmitter S1 is deactivated for the reflection measurement and the transmitter S2 is activated for the reflection measurement. Both measurements should be done immediately in succession.

The apparatus according to the present invention allows for a non-invasive, continuous detection of hemolysis in whole blood that is independent of hematocrit and oxygen saturation. It is characterized by a simple hardware setup and easy analysis of the measured results. The apparatus according to the present invention can be used in any blood treatment apparatus with an extracorporeal blood circulation. For quality assurance purposes, it is possible to use the apparatus according to the present invention also for detecting hemolysis in units of stored blood. To this end, the receiving unit can be configured for accommodating a unit of stored blood or a hose line on the unity of stored blood.

Another aspect of the present invention is the fact that a correction factor is established for the measurement of hematocrit using a conventional optical hematocrit measuring method, where the influence of the hemolysis is not taken into account.

FIG. 6 shows the dependent relationship between the hematocrit Hkt that is determined by means of the different measuring methods (RFₓ₌₅, reflection (x=5 mm), SSₓ₌₅, lateral scatter (x=5 mm), SSₓ₌₀, lateral scatter (x=0 mm), TSₓ₌₀, transmission (x=0 mm)) and the hemoglobin concentration in plasma PHb for a starting value of hematocrit of 36%, at which the hemoglobin concentration in plasma PHb is low. The error of the hematocrit measurement increases parallel to the increasing concentration of the free hemoglobin in plasma. The curve R shows the actual hematocrit for this series of measurements that was determined from a reference measurement.

It has been shown that the gradient of the curves remains the same if the starting value for the hematocrit, which is at 36% in FIG. 6, varies within a relatively wide range. The curve for a starting value other than 36% can easily be established by shifting the curve for the starting value of 36% in parallel with the new starting value.

Therefore, changing the starting value for the hematocrit results only in the curves being shifted in parallel. As a result, the difference between the measured values Hkt_{RFL}−Hkt_{TRM} that are obtained using the reflection and transmission measurement establishes the free hemoglobin in plasma (X-coordinates) substantially independently of the starting value.

FIG. 6 shows, for the reference curve R, a dependent relationship of the hemoglobin concentration in plasma PHb. However, for volumetric measurements of the blood of the patient, the reference curve R is, in reality, independent of free hemoglobin, which does not have a volume fraction. The dependent relationship of R(PHb), which is shown in FIG. 6, can be explained such that the shown curves result from a laboratory experiment in which hemolyzed whole blood is added to the sample. This whole blood consists of free hemoglobin and blood plasma, and therefore the proportion of the hematocrit in the sample is changed overall owing to the increased proportion of plasma. However, the distance between the curves also remains unchanged in patient measurements. The actual curve shape that would be produced in patient measurements is therefore the curve shape shown in FIG. 6, in which all the curves are rotated anticlockwise about their common left-hand starting point such that the reference curve R forms a horizontal line, and therefore Hkt is independent of PHb. However, this is irrelevant for determining the correction value.

The empirically established group of curves in FIG. 6 is stored in the computing and analyzer unit 16, preferably only for one starting value of the hematocrit Hkt, for example Hkt=36%, or for a large number of starting values. However, even if the group of curves is stored only for one starting value of the hematocrit Hkt, the hemoglobin concentration in plasma PHb can be derived clearly from the difference between the measured values Hkt of the hematocrit, since the plasma hemoglobin concentration depends substantially only on the difference between the measured values.

It is assumed that a hematocrit Hkt of 40% is measured using the reflection measurement (RFₓ₌₅) Hkt_{RFL}, and a hematocrit Hkt of 29.5% is measured using the transmission measurement (TSₓ₌₀). A hemoglobin concentration in plasma PHb of 2500 mg/dl is produced for both measurements (FIG. 6). The actual hematocrit (31%) can then be measured using the reference measurement, in which the hematocrit Hkt is 31% for a hemoglobin concentration in plasma PHb of 2500 mg/dl. The measured values for the hematocrit of 40% and 29.5% must be corrected, therefore, by 9% and −1.5%, respectively, which corresponds to the distance between the curve for the reflection measurement (RFₓ₌₅) and the curve for the reference measurement (R), or the distance between the curve for the transmission measurement (TSₓ₌₀) and the curve for the reference measurement (R). Both measurements thus result in correction factors of 9% and −1.5%, respectively. These correction factors are dependent on the hemoglobin concentration in plasma PHb and become smaller when the hemoglobin concentration in plasma PHb decreases.

1-17. (canceled)

18. A method for measuring hemolysis in whole blood or determining a correction factor for correcting the influence of hemolysis on a hematocrit measurement, the method comprising:
irradiating the whole blood with a radiation that is directed on the whole blood;
detecting a first emerging radiation with regard to the incoming radiation from a first direction for taking a first measurement according to a first measuring method;
detecting a second emerging radiation with regard to the incoming radiation from a second direction for taking a second measurement according to a second measuring method;
determining a first hematocrit value based on the intensity of the first emerging radiation emerging from the first direction according to the first measuring method; and
determining a second hematocrit value based on the intensity of the second emerging radiation emerging from the second direction according to the second measurement;
wherein the hemolysis or the correction factor for correcting the influence of the hemolysis on the hematocrit measurement is established on the basis of the first hematocrit value and the second hematocrit value.

19. The method according to claim 18, wherein the hemolysis or the correction factor for correcting the influence of the hemolysis on the hematocrit measurement is determined on the basis of the difference between the first hematocrit value and the second hematocrit value.

20. The method according to claim 19, wherein a hemolysis rate is determined on the basis of a preset linear relationship between the difference of the hematocrit value and the hemolysis rate detected according to the first and second measuring methods.

21. The method according to claim 18, wherein the first emerging radiation emerging from the first direction is the scattered radiation that was scattered in the whole blood.

22. The method according to claim 21, wherein the first emerging radiation emerging from the first direction is the backscattered radiation that is directed in the opposite direction of the direction of the radiation that is directed onto the whole blood.

23. The method according to claim 18, wherein the second emerging radiation emerging from the second direction is the transmitted radiation that passes through the whole blood and that is directed in the same direction as the radiation that is directed onto the whole blood.

24. An apparatus for measuring hemolysis in whole blood or for determining a correction factor that corrects the influence of hemolysis on a hematocrit measurement, the apparatus comprising:
a transmitting and receiving unit configured to irradiate the whole blood with a radiation that is directed onto the whole blood, and to detect a radiation that emerges in relation to the incoming radiation from a first direction for taking a first measurement according to a first measuring method, and to detect a radiation that emerges in relation to the incoming radiation from a second direction for taking a second measurement according to a second measuring method; and
a computing and analyzer unit configured to determine a first hematocrit value based on the intensity of the radiation emerging from the first direction according to the first measuring method, and to determine a second hematocrit value based on the intensity of the radiation emerging from the second direction according to the second measuring method;
wherein the computing and analyzer unit is configured to establish the hemolysis or the correction factor for measuring the influence of the hemolysis on the hematocrit measurement on the basis of the first hematocrit value and the second hematocrit value.

25. The apparatus according to claim 24, wherein the computing and analyzer unit is configured to determine the hemolysis or the correction factor for correcting the influence of the hemolysis on the hematocrit measurement on the basis of the difference between the first hematocrit value and the second hematocrit value.

26. The apparatus according to claim 25, wherein the computing and analyzer unit is configured to determine a hemolysis rate on the basis of a preset linear relationship between the difference of the hematocrit values and the hemolysis as detected according to the first and the second measuring methods.

27. The apparatus according to claim 24, wherein the transmitting and receiving unit is configured to measure the scattered radiation that is scattered in the whole blood according to the first measuring method.

28. The apparatus according to claim 27, wherein the transmitting and receiving unit is configured to measure the backscattered scattered radiation according to the first measuring method, and the backscattered scattered radiation is directed opposite relative to the direction of the radiation that is directed in the direction of the whole blood.

29. The apparatus according to claim 24, wherein the transmitting and receiving unit is configured to measure the transmitted radiation that passes through the whole blood according to the second measuring method, and the transmitted radiation is directed in the direction of the radiation that is directed onto the whole blood.

30. The apparatus according to claim 24, further comprising a unit with a receptacle for the clamped-like receiving of a transparent hose line for the whole blood, or a unit for mounting a cartridge that includes a channel for the whole blood.

31. The apparatus according to claim 30, wherein the transmitting and receiving unit includes:
a transmitter that is disposed on the one side of the hose line or the channel of the cartridge for irradiating the hose line or the channel of the cartridge with a radiation in the direction of an axis that runs vertically relative to the longitudinal axis of the hose line or the channel;
a first receiver that is disposed on the same side as the transmitter for receiving a scattered radiation in the direction of a measurement axis that runs vertically relative to the longitudinal axis of the hose line or the channel and that is disposed in the longitudinal direction of the hose line or the channel of the transmitter observing a spacing (x); and
a second receiver that is disposed on the other side of the hose line or the channel for receiving transmitted radiation in the direction of the axis that is located on the axis of the radiation of the transmitter that is directed to the hose line or the channel.

32. The apparatus according to claim 30, wherein the transmitting and receiving unit includes:
a first transmitter that is disposed on the one side of the hose line or the channel of the cartridge for irradiating the hose line or the channel of the cartridge with a radiation in the direction of an axis running vertically in the direction of the longitudinal axis of the hose line or the channel;
a second transmitter that is disposed on the other side of the hose line or the channel for irradiating the hose line or the channel with a radiation in the direction of an axis running vertically in relation to the longitudinal axis of the hose line or the channel, wherein the axis of the second transmitter is disposed observing a spacing (x) relative to the axis of the first transmitter in the longitudinal direction of the hose line or the channel; and
a receiver that is disposed on the same side as the second transmitter for receiving a backscattered radiation from the first transmitter and a transmitted radiation from the second transmitter, wherein the axis of the receiver lies on the axis of the first transmitter.

33. The method according to claim 18, wherein the radiation is light in the visible range of between 380 nm and 780 nm.

34. The apparatus according to claim 24, wherein the radiation is light in the visible range of between 380 nm and 780 nm.

35. An extracorporeal blood treatment apparatus with an extracorporeal blood circulation, the extracorporeal blood treatment apparatus including a blood supply line leading to an exchange unit, which is divided by a semi-permeable membrane into a first chamber and a second chamber, and including a blood return line that branches off from the first chamber of the exchange unit, wherein the extracorporeal blood treatment apparatus includes the apparatus according to claim 24.
